(19) United States
(12) Patent Application Publication
(10) Pub. No.: US 2026/0229924 A1
(43) Pub. Date: Aug. 6, 2026

(54) MEDICAL IMPLANT SYSTEM INCLUDING BASE STATION AND MEDICAL IMPLANT

(71) Applicant: NYXOAH S.A., Mont-St-Guilber (BE)

(72) Inventors: Carsten Müeller, Erfurt (DE); Timo Koch, Schmelz (DE)

(21) Appl. No.: 19/572,353

(22) Filed: Mar. 19, 2026

Related U.S. Application Data
(63) Continuation of application No. 18/647,050, filed on Apr. 26, 2024.
Application No. 18/647,050 is a division of application No. 15/533,089, filed on Nov. 30, 2015, now abandoned.

(30) Foreign Application Priority Data
Dec. 5, 2014 (DE) 102014118040.2

(51) Int. Cl. H02J 50/12; A61B 1/00; A61B 5/00; A61N 1/36; A61N 1/378; H02J 50/80; H02M 1/00; H02M 3/00; H02M 3/335; H02M 7/48; H03J 3/02

(52) U.S. Cl. CPC H02J 50/12; A61B 1/00016; A61B 1/00029; A61B 5/0031; A61N 1/36125; A61N 1/3787; H02J 50/80; H02M 3/01; H02M 3/015; H02M 3/33571; H03J 3/02; H02M 1/0058; H02M 7/4818; Y02B 70/10

(57) ABSTRACT

Exemplary embodiments relate to a control circuit for a base station for transmitting energy to a receiver by means of an electric resonant circuit. The control circuit comprises an evaluation device which is designed to compare energy that has been transmitted to a receiver resonant circuit of the receiver with an energy set value. The control circuit is designed to alter the energy input into the receiver resonant circuit of the receiver by altering a resonant frequency of the resonant circuit on the basis of the result of the comparison.

### CROSS-REFERENCE TO RELATED APPLICATIONS

This application is a continuation of U.S. patent application Ser. No. 18/647,050 filed on Apr. 26, 2024, which is a divisional application of U.S. patent application Ser. No. 15/533,089 filed on Dec. 21, 2017, which is a U.S. Nationalization of PCT International Application No. PCT/EP2015/078104 filed on Nov. 30, 2015, which claims priority to German Patent Application No. 10 2014 118 040.2 filed on Dec. 5, 2014 the disclosure of each of which is incorporated herein, in its entirety, by this references.

### TECHNICAL AREA

Execution examples deal with a control circuit for a base station for transferring energy to a receiver, preferably a medical implant, by means of an electric resonant circuit and an evaluation device for determining energy transferred from an electric resonant circuit of a base station to a receiver resonant circuit of a medical implant.

### BACKGROUND

In the area of the medicine technology, implants are used in many cases. In the operation of such implants, e.g., for the stimulation of nerves or muscles in the body, it can be helpful to get information about a stimulation parameter, which appears at one point of the stimulation. For instance, it can be important to know the strength of a stimulation power at the place, in order to be able to estimate and configure an effect of the power. Further, a functional check of the implant can be necessary during or immediately after an implantation, what is executable, however, up to now only under complicated conditions.

In case of energy transfer to implants, resonance amplifiers are widespread, which can enable a simplified structure and a simplified impact. However, it can happen in many cases that a resonant circuit of the implant and a resonant circuit of a primary side (base station outside the body) have changed their position or alignment to each other, which can make energy transfer ineffective or even impossible.

In some cases, for instance, in case of a battery operation, an operating voltage can be relatively low. Since, e.g., use of a switch DC-voltage converter can possibly lead to interference radiation, it can be sensible to adapt the power output stages directly to a battery voltage, so that the DC-voltage converter can be omitted if possible. A DC-voltage converter can cause an unfavourable influence on energy consumption, interference radiations or problems during an integration of individual functional units with an only restricted construction volume.

Furthermore, high voltages can also appear due to resonance super-elevations (e.g., 2000 Vᵢₙ a series resonant circuit). Use of the semi-conductor construction elements, which can be used in this voltage range, cannot be often possible here just like that. The semi-conductor construction elements, which can be used for switching on of the type of high voltages and powers, are laid out, in many cases, for an area of the power electronics with a frequency of 50 Hz, and can therefore be too big for using in medical implants or can be problematic in relation with a actuation time or necessary control power.

Besides, depending on a frequency range (e.g., 8 MHz), parasitic elements like capacities of construction elements can become apparent as interfering, what can complicate selection of a construction element, for instance, switching transistors. Furthermore, protective circuits can appear negative against overload due to too high voltages or powers, which are enclosed, for instance, by integrated construction elements, by consumption of useful energy in the resonant circuit.

According to a conventional solution, a stimulation effect or a functional efficiency of an implant can be ascertained, when the stimulation is estimated with the help of an induced body reaction, for instance, a muscle contraction. Here, in case of implants without energy storage, the procedure can be begun with relatively low stimulation energy, and a desired effect can be configured with the help of an observed reaction of a patient. Further, transfer or sending from the implant is possible with customary procedures; however, it can be necessary here to provide the energy necessary for this to the implant by means of energy storage.

According to another conventional solution, the energy to be transferred can be regulated by different amplifier concepts (amplifier of the classes D or E). Hereby, customary methods, like for example, a change in a duty cycle can be applied while controlling an output stage, controlling with signals of different levels or a regulation of the operating voltage. Also in other areas of the technology, in which induction-operated circuits are used, such problems can be at least partially relevant.

It is worthwhile to create an improved concept for a power transfer in a system from base station and medical implant and for monitoring parameters of the medical implant.

### SUMMARY

According to the first aspect, execution examples refer to a control circuit for a base station for transferring energy to a receiver by means of an electric resonant circuit. The control circuit encloses an evaluation device, which is designed to compare energy transferred to a receiver resonant circuit of the receiver with a target energy value. The control circuit is also designed to cause a changed energy entry in the receiver resonant circuit of the receiver by a change in a resonance frequency of the resonant circuit based on a result of the comparison. By this it can be avoided, under circumstances, saving energy, and thereby possibly also an appearance of too high voltages. In other words, increase in a transferred energy by approximating resonance frequencies of the resonant circuits can be attained hereby, and increasing an input power for this can be possibly omitted. Further, a component volume and production costs can be possibly reduced.

In some execution examples, the receiver is a medical implant.

In some execution examples, the control circuit is designed to counteract against a change in a coupling of the resonant circuit in the receiver resonant circuit at least partially by changing the resonance frequency of the resonant circuit. As mentioned, increasing an output stage power can thereby be omitted, and an appearance of high voltages can be avoided.

In some execution examples, the control circuit is designed to approximate the resonance frequency of the resonant circuit to an internal frequency of the receiver resonant circuit, if the transferred energy is lesser than the target energy value. This can cause an increased efficiency of energy transfer.

In some execution examples, the control circuit is designed to remove the resonance frequency of the resonant circuit from an internal frequency of the receiver resonant circuit, if the transferred energy is more than the target energy value.

In some execution examples, the control circuit is designed to cause change in the resonance frequency by changing an electric resistance in the resonant circuit.

In some execution examples, the control circuit is designed to cause change in the electric resistance by varying an excitation current. Here, the electric resistance is an effective resistance of the resonant circuit.

In some execution examples, the control circuit is designed to change the electric resistance within a predetermined time interval, whereby the time interval is smaller than an oscillation period of the excitation current.

In some execution examples, the control circuit further encloses a series connection of a first electric component and a second electric component. The resonant circuit is coupled with an electro-conductive connection between the first electric component and the second electric component, so that the excitation current is activated by a first input signal of the first electric component and/or a second input signal of the second electric component. This can enable an active control of the control circuit.

In some execution examples, the control circuit further encloses at least one first voltage source. The first voltage source is designed to generate the first input signal in such a way that the first input signal shows a rising or falling course alternatively in the time interval and a constant course in another time interval. By this, changing the effective resistance of the resonant circuit can be caused during the time interval.

In some execution examples, a ratio of the time interval to the other time interval is smaller than 1. By this, a change in the resistance can be possibly caused, so that an influence on a resonant circuit quality is thereby reduced.

In some execution examples, the control circuit is designed to regulate a ratio of the time interval to the other time interval.

In some execution examples, the control circuit is designed to reduce the resonance frequency by increasing the ratio of the time interval to the other time interval or increase the resonance frequency by reducing the ratio of the time interval to the other time interval.

In some execution examples, the control circuit further encloses at least one second voltage source. The second voltage source is designed to generate the second input signal in such a way that the second input signal shows a rising or falling course alternatively in the time interval and a constant course in the other time interval. Here, the rising course of the first input signal coincides with the falling course of the second input signal or the rising course of the second input signal coincides with the falling course of the first input signal.

In some execution examples, the first electric component is a first amplifier, the second electric component is a second amplifier, the first input signal is a first input voltage and the second input signal is a second input voltage.

In some execution examples, the first electric component is a first transistor, the second electric component is a second transistor, the first input signal is a first control voltage and the second input signal is a second control voltage.

In some execution examples, the control circuit further encloses a series connection of a third electric component and a fourth electric component. Here, the resonant circuit couples to a bridging branch between an electro-conductive connection between the first electric component and the second electric component and an electro-conductive connection between the third electric component and the fourth electric component, so that the excitation current is activated by the first input signal, the second input signal, a third input signal of the third electric component and/or a fourth input signal of the fourth electric component.

In some execution examples, the control circuit further encloses a resistive element coupled with the resonant circuit with a temporally variable resistivity. The resistive element encloses a control connection for receiving a control signal for changing the resistivity.

In some execution examples, the control circuit further encloses an evaluation device. The evaluation device is designed to determine the transferred energy based on a modulation characteristic of a recoupling signal coupling with the electric resonant circuit.

Some execution examples are directed to a base station with a given control circuit. Here, the resonant circuit is designed to receive a recoupling signal with a modulation characteristic, which contains information about energy transferred to the receiver resonant circuit of the receiver.

In some execution examples, the receiver is a medical implant.

Some execution examples refer to a system, which encloses a receiver and a given base station. The receiver is designed to receive the energy signal and to send the recoupling signal.

According to another aspect, execution examples refer to an evaluation device for determining energy transferred from an electric resonant circuit of a base station to a receiver resonant circuit of a medical implant. The evaluation device encloses an analyser, which is designed to determine a modulation characteristic of a signal appearing in the electric resonant circuit of the base station, and to determine the energy transferred to the receiver resonant circuit based on the modulation characteristic. By this, a recoupling effect of the medical implant on the base station can be caused, with which an internal energy supply of the implant can be omitted. An access to information about a stimulation effect or a functional efficiency of the implant can be thus possibly simplified.

In some execution examples, the analyser further encloses a demodulator. The demodulator is designed to determine the modulation characteristic of the signal by demodulation.

In some execution examples, the modulation characteristic of a frequency corresponds to an amplitude modulation of the signal.

In some execution examples, the demodulator is designed to determine an envelope curve of the signal. The modulation characteristic corresponds to the frequency of the envelope curve.

In some execution examples, the evaluation device is designed to determine the modulation characteristic during the transfer of the energy.

In some execution examples, the modulation characteristic of a frequency change corresponds to a frequency modulation of the signal.

In some execution examples, the demodulator is designed to measure a first frequency of the signal at a first time point and a second frequency of the signal at a second time point following the first time point. Here, determining the modulation characteristic includes determining a difference of the first frequency and the second frequency.

In some execution examples, the evaluation device is designed to determine the modulation characteristic immediately after the transfer of the energy.

Some execution examples refer to a medical implant, which encloses the receiver resonant circuit and a rectifier circuit. The rectifier circuit is or encloses hereby a capacity diode. This can enable a rectification of an induced voltage.

Some execution examples refer to a base station. The base station encloses an electric resonant circuit for transferring energy to a receiver resonant circuit of a medical implant and a given evaluation device.

In some execution examples, the base station further encloses a resonance amplifier coupled with the electric resonant circuit. The resonance amplifier is designed to change a resonance frequency of the electric resonant circuit based on a control signal. Here, the analyser is designed to generate the control signal based on the ascertained energy.

Some execution examples refer to a system, which encloses a medical implant and a given base station.

According to still another aspect, execution examples refer to a procedure for determining energy transferred from an electric resonant circuit of a base station to a receiver resonant circuit of a medical implant. The procedure encloses determining a modulation characteristic of a signal appearing in an electric resonant circuit of the base station. Besides, the procedure encloses determining energy transferred to the receiver resonant circuit based on the modulation characteristic. By this, it can be made possible to use an echo signal appearing during a power transfer for gaining parameters appearing in the implant. Here, an internal energy supply to the implant can possibly be omitted.

According to still another aspect, execution examples refer to a procedure for transferring energy to a medical implant by means of an electric resonant circuit. The procedure encloses comparison of energy transferred to a receiver resonant circuit of the medical implant with a target energy value. Besides, the procedure encloses causing a change in energy entry of the resonant circuit in the receiver resonant circuit of the medical implant by a change in the resonance frequency of the resonant circuit based on a result of the comparison. This can enable an improved reaction to interferences during energy transfer, like for example, a position change of base station and implant, by an intended regulation of the resonance frequency of the resonant circuit.

In some execution examples, the procedure optionally encloses determining the energy based on a modulation characteristic of a recoupling signal coupling with the electric resonant circuit.

In addition, other execution examples also create a programme or computer programme with a programme code to execute one of the given procedures, when the programme code is entered in a computer, a processor or a programmable hardware component, like for example an integrated circuit specific for application (ASIC).

Although some execution examples were described with the help of an example of a medical implant, execution examples are not limited to medical implants, but can be transferred to a huge number of technical devices, which are designed to receive energy.

### SHORT DESCRIPTION OF FIGURES

Execution examples are explained in details below with reference to the enclosed figures. The following are shown:

FIG. 1 a simplified diagram of a conventional resonant circuit;

FIG. 2 a block diagram of a control circuit for a base station for transferring energy to a medical implant by means of an electric resonant circuit according to an execution example,

FIG. 3 a diagram of an electric resonant circuit according to an execution example;

FIG. 4 temporal courses of two input signals, an effective resistance and an excitation voltage according to an execution example;

FIG. 5 a procedure for transferring energy to a medical implant by means of an electric resonant circuit according to an execution example;

FIG. 6 a diagram of a resonant circuit of a medical implant with a capacity diode according to an execution example;

FIG. 7 a simplified schematic structure of an evaluation device according to an execution example;

FIG. 8 a detailed schematic structure of an evaluation device according to an execution example;

FIG. 9 temporal courses of a primary signal, an echo signal and a modulation characteristic of the echo signal according to an execution example, and

FIGS. 10a, b temporal courses of a customary voltage impulse and a voltage impulse according to an execution example;

FIG. 11 a procedure for determining energy transferred from an electric resonant circuit of a base station to a receiver resonant circuit of a medical implant according to an execution example.

### DESCRIPTION

Now different execution examples are described with more details with reference to the enclosed drawings, in which some execution examples are shown. In the figures, the thickness dimensions can be shown with excessive dimensions by lines, layers and/or regions for the want of clarity.

In the following description of the enclosed figures, which show only some exemplary execution examples, the same reference signs can depict the same or similar components. Further, summarised reference signs can be used for components and objects, which appear several times in an execution example or in a drawing, are, however, described with respect to one or several features together. Components or objects, which are described with the same or summarised reference signs can be given with respect to individual, several or all features, for instance, of their dimensions, in the same way, however, if necessary also differently, provided that something else does not arise explicitly or implicitly from the description.

Although execution examples can be modified and changed in a different way, execution examples in the figures are shown as examples and are described there in detail. However, it is clarified that it is not intended to limit the execution examples to the respectively published forms, but that the execution examples should rather cover all functional and/or structural modifications, equivalents and alternatives, which lie in the area of the invention. The same reference signs depict the same or similar elements in the entire figure description.

It should be noted that an element, which is given as “linked” or “coupled” with another element, can be directly linked or coupled with the other element or that there can be intermediate elements. If, on the other hand, an element is given as “directly linked” or “directly coupled” with another element, then there are no intermediate elements. The other terms, which are used to describe the relation between elements, should be interpreted in a similar way (e.g., “between” as against “directly in between”, “adjoining” as against “directly adjoining” etc.).

The terminology, which is used here, serves only the description of certain execution examples and should not limit the execution examples. As used here, the singular form “a/an” and “the” should contain also include the plural forms, as long as the context does not mention something else clearly. Further, itis clarified that the expressions like, for example, “contains”, “containing”, “shows” and/or, “showing”, as used here, state the presence of given features, integers, steps, work procedures, elements and/or components, but do not exclude the presence or the addition of one or several features, integers, steps, work procedures, elements, components and/or groups thereof.

As long as nothing else is defined, all concepts used here (including technical and scientific terms) have the same meaning, which is given to them by an average expert in the area, to which the execution examples belong. Further, it is clarified that the expressions, e.g., those, which are defined generally used dictionaries, are to be interpreted as if they had the meaning, which is consistent with their meaning in the context of the appropriate technology, as long as this is not defined expressively differently here.

Execution examples can enclose a parametric amplifier or a resonant circuit. By execution examples, it is aimed to attain regulation and tracking of an internal frequency of an amplifier output stage and a resonant circuit quality.

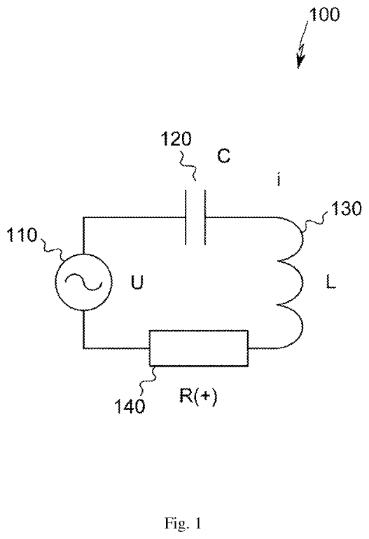

FIG. 1 shows a conventional structure of a resonant circuit 100 according to a customary example. The resonant circuit 100 encloses, at first, a voltage source 110, which provides an alternating voltage. A condenser 120 is coupled with the voltage source 110. A coil 130 is coupled with the condenser 120. By coupling the coil 130 with the voltage source 110, a closed circuit is formed. An Ohm resistance 140 can serve, for instance, an overall presentation of resonant circuit losses. Here, the resistance 140 (R), a capacity C of the condenser 120 and an inductance L of the coil 130 can be temporally variable. In closed power circuit, there falls a voltage

$$U = {U}_{C} + L \frac{d i}{d t} + iR ( t )$$

in the resonant circuit, which causes flowing of an induction current

$$i = C \frac{{dU}_{C}}{dt}$$

With temporally constant loss resistance R (t)=R, the resonant circuit can be described by the differential equation (DGL 1):

$$\frac{1}{L} \frac{dU}{dt} = \frac{1}{LC} i + \frac{R}{L} \frac{\partial i}{\partial t} + \frac{{\partial }^{2} i}{\partial {t}^{2}}$$

Here, the terms are derived, which characterize a frequency of the resonant circuit

$${ω}_{1} = \frac{1}{\sqrt{LC}}$$

And an attenuation.

If the resistance R(t), which stands for a description of total losses, is applied as dependent on time, then an extended differential equation (DGL 2) is derived by additional terms:

$$\frac{1}{L} \frac{dU}{dt} = ( \frac{1}{LC} + \frac{1}{LC} \frac{\partial R ( t )}{\partial t} ) + \frac{R ( t )}{L} \frac{\partial i}{\partial t} + \frac{{\partial }^{2} i}{\partial {t}^{2}}$$

By comparing the terms in DGL 1 with those in DGL 2, the following appears in a coefficient for a circuit frequency of the resonant circuit

$${ω}_{2} = \sqrt{\frac{1}{L C} + \frac{1}{L} \frac{\partial R ( t )}{\partial t}}$$

with time-related resistance, an additional term ∂R(t)/∂t. Now, the term, which characterizes the attenuation, is also time-related here. Hereby, with respect to the attenuation and a resonant circuit quality Q=ωL/R, an exact temporal course of the resistance change can be, on one hand, unimportant, on the other hand, an integral resistance over a vibration period can have influence on this characteristics; for instance, increase in the integral of the resistance can lead to reduction in the resonant circuit quality Q. Explained once again in other words, the resonance frequency ω₂ and the quality Q of the resonant circuit are determined by the capacity C, the inductance L and the resistance R(t).

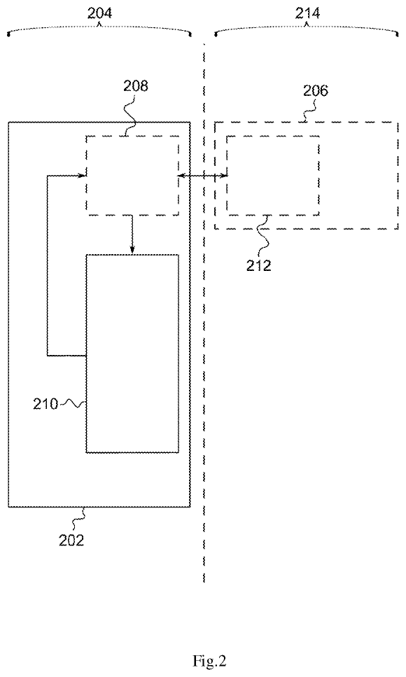

FIG. 2 shows a control circuit 202 for a base station 204 for transferring energy to a receiver 206 by means of an electric resonant circuit 208 according to an execution example. Only optionally available elements are shown, here, with stroked lines or boxes. The control circuit 202 encloses an evaluation device 210, which is designed to compare energy transferred to a receiver resonant circuit 212 of the receiver 206 with a target energy value. The control circuit 202 is further designed to cause a changed energy entry in the receiver resonant circuit 212 of the medical implant 206 by a change in a resonance frequency of the resonant circuit 208 based on a result of the comparison. The target energy value can be derived, e.g., from a target power value.

In some execution examples, the receiver 206 is a medical implant 206. The receiver 206 is illustrated below as a medical implant 206, what is to be understood, however, only exemplarily. In other execution examples, any other type of a receiver, which encloses a receiver resonant circuit 212, can be used alternatively for the medical implant 206.

Here, the medical implant 206 is located in a body 214 of a patient. When the base station 204 approaches the body 214 of the patient, an interaction can take place between the resonant circuit 208 and the receiver resonant circuit 212, or expressed in other words, a power exchange or an inductive coupling. The resonant circuit 208 can be integrated, hereby, in the control circuit 202 or also coupled in another way with this. Thus an exchange of signals or information can be enabled between the resonant circuit 208 and the evaluation device 210. In some execution examples, the control circuit 202 is further designed to counteract against a change in a coupling of the resonant circuit in the receiver resonant circuit by changing the resonance frequency of the resonant circuit 208 at least partially. A transferred power can be thereby regulated in a way, which can be more energy-efficient than increasing a transmission power.

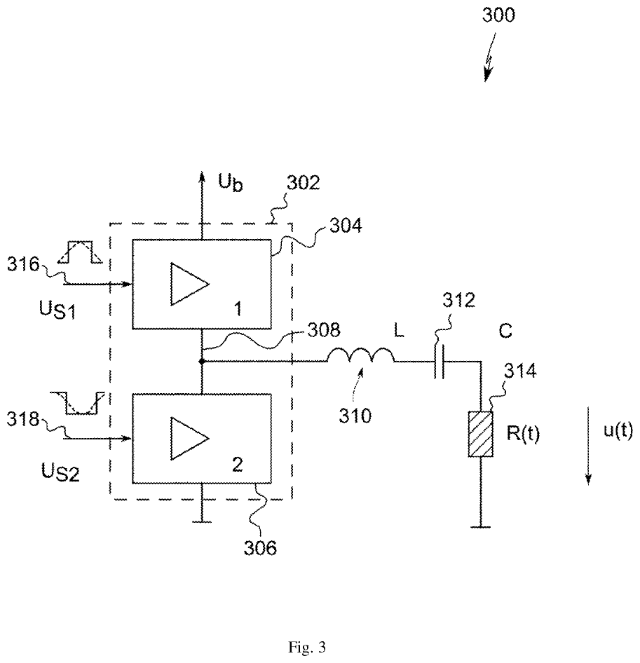

An execution example of a resonant circuit 300 is shown in FIG. 3. The resonant circuit 300 can, for instance, be identical with the resonant circuit 208 (refer to FIG. 2). Further, the resonant circuit 300, as FIG. 3 shows, can be explained as a series resonant circuit, with other execution examples, however, also as parallel resonant circuit. The resonant circuit 300 is coupled to a half bridge 302 or series connection of a first electric component 304 and a second electric component 306, or is enclosed by the half bridge 302. Exactly said, the first electric component 304 is coupled between a supply voltage U_{b} and an electro-conductive connection 308, and the second electric component 306 is coupled between the electro-conductive connection 308 and mass. Between the connection 308 and mass, a series connection of an inductive element 310 (e.g., of a coil) and a capacitive element 312 (e.g., of a condenser) is made. A time-related resistance 314 R(t) represents here appearing energy losses in the resonant circuit 300. A first input signal 316 is supplied to the first electric component 30, and a second input signal 318 is supplied to the second electric component 306. The first input signal 316 and the second input signal 318 can be provided by a control device. In the execution example shown here, the first electric component 304 is a first amplifier, the second electric component 306 is a second amplifier, the first input signal 316 is a first input voltage Uₛ₁, and the second input signal 318 is a second input voltage Uₛ₂. Different classes of amplifiers can be used here. However, with an amplifier of the class E, a coupling factor of a transmitter coil and a receiver coil can have considerable influence on operating qualities of the amplifier. With the application in medical implants, in which there can often be position changes of the coils to each other, this can be controlled only with increased expenditure. This can, however, mean that an increased efficiency is increased again with this amplifier type. Hence, in some execution examples, amplifiers of the class D are also used. In another execution example, the first electric component 304 can be a first transistor (e.g., field effect transistor), the second electric component 306 is a second transistor, the first input signal 316 is a first gate or control voltage, and the second input signal 318 is a second gate or control voltage. By changing the gate or control voltages, the conductivity of the electric components 304; 306 can be influenced, by which a time-related excitation voltage arises in the electro-conductive connection 308. The excitation voltage can cause a current i(t), which can cause a temporally variable excitation of the resonant circuit 300.

According to some execution examples, change in the resistance 314 in the resonant circuit 300 causes change in the resonance frequency or internal frequency of the resonant circuit 300. The resistance 314 can be an effective resistance of the resonant circuit 300. For instance, varying an excitation current can cause change in the electric resistance 314. The excitation current can be caused again by the first input voltage 316 (Uₛ₁) and the second input voltage 318 (Uₛ₂). Thus it is possible in some execution examples to approximate the resonance frequency of the resonant circuit 300 to an internal frequency of the receiver resonant circuit, if the transferred energy is smaller than the target energy value, or to remove from the internal frequency of the receiver resonant circuit, if the transferred energy is bigger than the target energy value.

It can be possible here, that the temporal derivation ∂R(t)/∂t of the resistance 314 influences the frequency, and a temporal average of the resistance 314 influences the quality Q=ωL/R of the resonant circuit (for this, refer also to FIG. 1). Thus a change in the quality factor Q of the resonant circuit 300 can also be connected with a periodical change in the resistance 314 in the resonant circuit 300, which can again be caused with a change in a received amplitude of the energy transferred from the resonant circuit 300 to the receiver resonant circuit. The principle described here can also serve to regulate the transferred energy. Consequently, it can be worthwhile to reduce the quality change below a predetermined limit value and to include in the regulation of the energy to be transferred. In some execution examples, this can be attained, when the resonant circuit 300 is coordinated with a frequency below the desired resonance frequency of the receiver resonant circuit. The quality Q can be a measure of vibration behaviour or resonant circuit losses of an excited resonant circuit. Here, increase in the quality Q can correspond to a reduction in a vibration attenuation. If increase in the loss resistance 314 appears in the temporal means at the same time as increase in the resonance frequency of the resonant circuit 300, then increase in an inductive resistance X_{L}=ωL can counteract in the equation Q=ωL/R of the increase in the resistance 314. Expressed in other words, the changes can occur in a common direction, by which the quality Q changes just slightly. With the change in the resonance frequency of the resonant circuit 300, in other words, increase in an amount of the resistance change ∂R(t)/ωt can have a considerable influence, and a defined temporal course of the resistance change can be negligible. By this, a large number of designs can be enabled, of which one is explained below in details.

In an execution example, the already described circuit arrangement can be used for amplifiers, or amplifier output stages (half bridge connection 302) or a full bridge connection, further also for a throttle (choke coil). A periodical change in the loss resistance 314 R(t) can be caused by several variations. In a first variation, the already available first amplifier 304 and second amplifier 306 are used. In a second variation, a switching element can be coupled additionally or alternatively with the resonant circuit. This can be, for instance, a resistive element with a temporally variable resistivity, which corresponds to the resistance 314. Here, a loss voltage u(t) marked by an arrow, which influences the excitation voltage, drops over the resistive element. The resistive element can enclose a control connection for receiving a control signal for changing the resistivity.

In the first variation, in which the half bridge connection 302 is used, a simplification in the structure can be possible. The control of the first amplifier 304 and the second amplifier 306 can be combined, for instance, with a control for a regular amplifier mode. In order to reach the desired resistance change ∂R(t)/∂t, e.g., a conductance of the switching edges of the input voltages Uₛ₁, Uₛ₂ can be varied for controlling the output stage, or phase displacements can be made in the input voltages. Changing the input voltages causes a temporally variable conductivity of the amplifiers 304, 306, and thus a time-related excitation voltages u(t), falling in FIG. 3 over the resonant circuit 300, which again causes an excitation current. The resistance 314 R(t) can be changed by this excitation current. In the second variation, positive voltages can possibly appear with the additional or alternative switching element (e.g., metal oxide semiconductor field effect transistor, MOSFET) only in a predefined area, for which optionally available, internal protective circuits are construed.

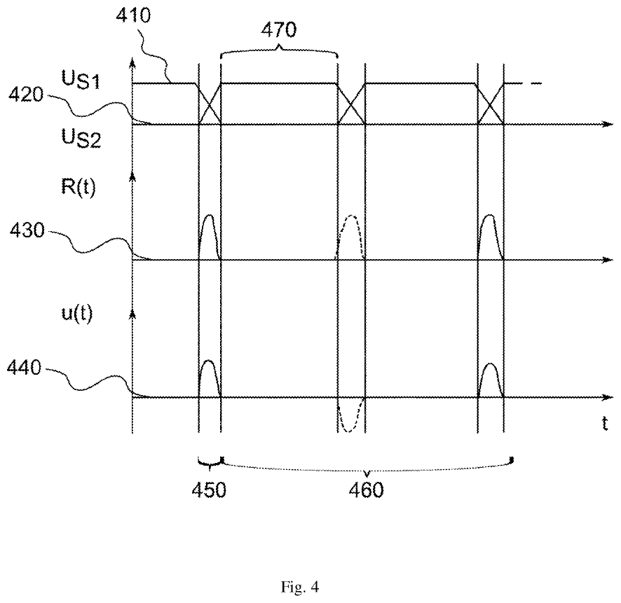

FIG. 4 shows the already explained connection s on the basis of different signal courses. Hereby, the temporal courses of the first input voltage Uₛ₁ (410), the second input voltage Uₛ₂ (420), the resistance R(t) (430) and the excitation voltage u(t) (440) are shown. Here, in an execution example, the control circuit is designed to change the resistance R(t) within a predetermined time interval 450. Here, the predetermined time interval 450 is smaller than a vibration period of the excitation current. Here, the vibration period of the excitation current can correspond to a vibration period 460 of the excitation voltage. The control circuit can further show a voltage source for generating of the first or second input voltage. Here, the voltage source can be designed to generate the first or second input voltage in such a way that this alternatively shows a rising or falling course in the time interval 450 and a constant course in another time interval 470. Here, a ratio of the time interval 450 to the other time interval 470 can be smaller than 1, what also FIG. 4 makes clear. Further, the control circuit can be designed to regulate the ratio of the time interval 450 to the other time interval 470. It can thus be made possible in some execution examples, to cause reduction in the resonance frequency by increasing the ratio of the time interval 450 to the other time interval 470 or increase in the resonance frequency by reducing the ratio of the time interval 450 to the other time interval 470. Expressed in other words, increasing the ratio causes reduction in the resistance change ∂R(t)/∂t, and according to the already given context

$${ω}_{2} = \sqrt{\frac{1}{L C} + \frac{1}{L} \frac{\partial R ( t )}{\partial t}}$$

Reduction in the resonance frequency ω₂, and vice versa.

Here, in some execution examples, constant temporal courses of the input voltages Uₛ₁, Uₛ₂ can coincide temporally. Besides, a rising course of the first input voltage Uₛ₁ can coincide temporally with a falling course of the second input voltage Uₛ₂ and vice versa. In other words, the first amplifier and the second amplifier are controlled counterphase with the respective input voltages. Here, the switching edges can be variable in the increase, what can be attained by temporary slowing down the switching time (this corresponds to a deliberate “worsening” of the switching qualities) of the amplifier stage. From it, a predetermined variable temporal course of the loss resistance R(t) (shown in FIG. 4 as drawn through and stroked curves) can arise during the switching process between the two amplifiers. The resistance course itself can cause a voltage course u(t) in the amplifier elements with positive and negative polarity. A similar effect can be attained, according to the second variation, with the additional discreet switching element with the behaviour of a temporally variable resistance. Here, the switching edges of the input voltages can remain unchanged. Here, according to phase position, it can be possible to receive only positive voltage superelevations (drawn through curve). Here, an easier control of the voltage superelevations can also be attained by using conventional construction elements. Expressed in other words, comparatively increased voltages can appear only in a polarity, what can cause, for instance, that body diodes enclosed by MOSFETs consume only few or no useful energy.

In an execution example, a choke can be used optionally for the half bridge connection. Further, in another execution example, the control circuit can enclose a series connection of a third electric component and a fourth electric component. Here, the resonant circuit is coupled with a bridging branch between an electro-conductive connection between the first electric component and the second electric component and an electro-conductive connection between the third electric component and the fourth electric component, so that the excitation current is activated by the first input signal, the second input signal, a third input signal of the third electric component and/or a fourth input signal of the fourth electric component. Expressed in other words, a full bridge connection can be used alternatively for the half bridge connection. The half bridge connection or the full bridge connection can be each designed with complementary steps or a High Side driver. Thus e.g. a 4-fold increased source power can be possibly attained related to the respectively same supply voltage. The switching element can be further used in combination with the half bridge or the full bridge.

Again explained in other words, the control circuit can be designed to regulate parameters of a resonant circuit in combination with an electronic control and corresponding software (microprocessor/computer). Here, a frequency-related source amplifier amplitude can be balanced as a result of different internal or resonance frequencies of the resonant circuit. Under circumstances, execution examples can enable an operation of resonance amplifiers with reduced, unstabilized supply voltage, for instance, outgoing from a battery. A source power or transferred energy can be thus regulated by controlling blind elements (reactances), what can cause a reduction in losses. A temporal course of a change in the quality Q (loss resistance) can hereby be arbitrary. In some execution examples, an amplifier characteristic can be adapted to relatively small, unstabilized supply voltages, what can possibly permit an improved use of supply voltages from batteries. Parameters can be configured, for instance, by software control. Hereby, an adaptation can be carried out to different requirements, whereby hardware changes can be possibly omitted. Here, undesirable effects of protective circuits (e.g., of the body diode of a MOSFET) can be possibly reduced.

The given execution examples can be used for different purposes, for instance, for a frequency tracking of resonant circuits, in which a change-over of reactances can possibly be omitted. Also a power adaptation or a control of an internal resonance of amplifier stages can be carried out. Some execution examples can also be implemented in connection with variable high-frequency amplifiers.

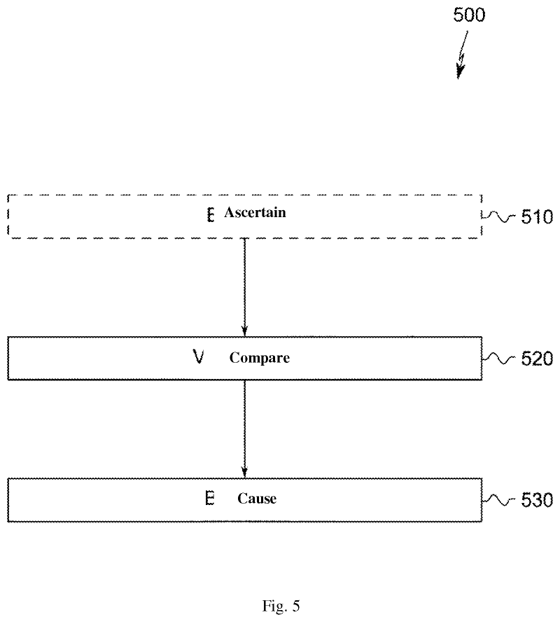

FIG. 5 shows a procedure 500 for transferring energy to a medical implant by means of an electric resonant circuit, according to an execution example. The procedure 500 encloses a comparison 520 of energy transferred to a receiver resonant circuit of the medical implant with a target energy value. Besides, the procedure 500 encloses an initiation 530 of a change in energy entry in the receiver resonant circuit of the medical implant by a change in the resonance frequency of the resonant circuit based on a result of the comparison. This can enable an improved reaction to interferences during energy transfer, like for example a position change of base station and implant, by a specific regulation of the resonance frequency of the resonant circuit.

In some execution examples, the procedure 500 encloses optionally a determination 510 of the energy based on a modulation characteristic of a recoupling signal coupling with the electric resonant circuit. Here, the determination 510 can precede the comparison 520.

Again explained in other words, execution examples can enable a regulation of the transferred energy amount during the operation. Here, compared to customary methods, dynamic losses can be reduced under circumstances. Besides, the magnetic energy to be transferred can be adapted in execution examples. Hereby, only one supply voltage source with unstabilised supply voltage can be used, if necessary, (battery operation), and thereby losses are possibly reduced. Besides, execution examples can permit a regulation of the internal frequency of resonant circuits. Requirements for construction elements, a number of construction elements, necessary construction space or also an influence of parasitic capacities of switching elements can be possibly reduced by this. By a parametric regulation according to execution examples, the voltage load can be reduced, under circumstances, for semi-conductor construction elements as a result of resonance superelevations. In addition, it can become possible with a setting of different resonance frequency that additional construction elements can be omitted.

Furthermore, the control circuit can enclose further, in some execution examples, an evaluation device. The evaluation device is designed to determine the transferred energy based on a modulation characteristic of a recoupling signal coupling with the electric resonant circuit. Optionally, the control circuit can also be enclosed by a base station. Further, the resonant circuit can be designed to receive the recoupling signal with the modulation characteristic. Here, the modulation characteristic can enclose information about energy transferred to the receiver resonant circuit of the medical implant. Even other execution examples refer further to a system, which encloses the given base station and the medical implant. The medical implant is designed to receive the energy signal and to send the recoupling signal. The terms “receive” and “send” can enclose here, in the broader sense, also other types of coupling besides the customary understanding, with which a data transfer can take place. For instance, the resonant circuit of the base station and the receiver resonant circuit of the medical implant can be coupled with each other also by modulated backscattering (load modulation).

According to conventional solutions, a functional way of passive implants is checked, e.g., for a nerve stimulation, with the help of applied reactions of a patient. However, this control mechanism can be subjective and also inexact, since only yes-no statements can be made there. In other words, the patient can react to the stimulation or not. Degenerative or ageing-related changes in the electronics of the implant can be ascertained in this manner only with complications. Raising more exact data of the parameters, which are in the implant, for instance, resonance frequency or stimulation intensity, or parameters belonging to body of the patient or test subject, for instance, tissue impedance (this can be a final impedance of the implant) can be, hereby, desirable. A conventional solution is the use of an active implant. However, additional construction space can be necessary, hereby, for an energy source.

In order to determine the energy transferred to the medical implant, several variations are suggested in context of execution examples. According to the first variation, a remaining energy can be used in the implant, which is available immediately after a stimulation impulse or another specific external excitation. The remaining energy can subside according to a predefined pattern. Here, electromagnetic vibrations with a characteristic frequency can be radiated. According to the second variation, energy, which is supplied to the implant during the stimulation, can be used. Hereby, a predetermined frequency, which is radiated during the stimulation, can be used.

In some execution examples, the medical implant encloses a diode. The diode can show a barrier layer capacity dependent on an adjacent barrier voltage. The energy subsiding after an end of the stimulation impulse in the implant (also called as a “trailing edge”) or the energy present during the stimulation impulse generates certain barrier voltage in the diode, e.g., subsiding with given time constants. This can lead to a frequency modulation of the radiated high frequency. During the radiation, this frequency can change, so that information about the fact which power i(t) has flowed or flows in a period of the trailing edge or in a course of the stimulation impulse.

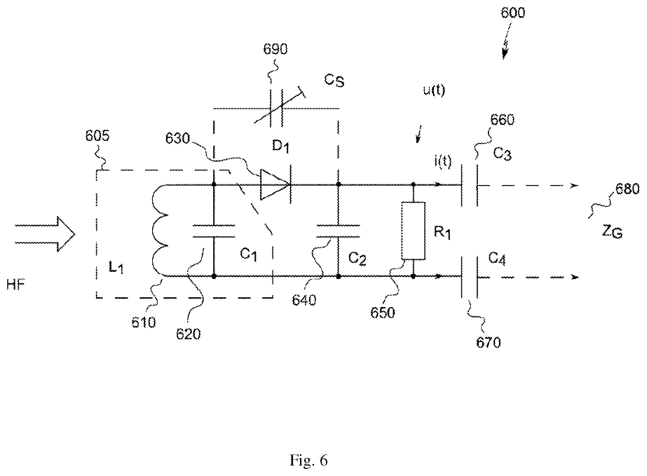

FIG. 6 shows a diagram for a possible execution example of a medical implant 600, which can serve for an electric stimulation of muscles or nerves. A parallel connection of an inductive element 610 with a first capacitive element 620 forms a parallel resonant circuit 605. Here, the inductive element 610 shows an inductance L₁, and the first capacitive element 620 shows a capacity C₁. With a diode 630, a coupling point of the first capacitive element 620 is connected with a coupling point of a second capacitive element 640, and another coupling point of the first capacitive element 620, which is averted to the coupling point, is connected with another coupling point of the second capacitive element 640. Here, the second capacitive element 640 shows a capacity C₂. Here, a barrier direction of the diode 630 points from the second capacitive element 640 to the first capacitive element 620. In some execution examples, the diode 630 is a rectifier diode, or expressed in other words, is enclosed by a rectifier circuit, and can cause a rectification of a high-frequency voltage received from outside the body (e.g., 8 MHz), from which a stimulation impulse can be formed. Here, the diode 630 can be a capacity diode. In FIG. 6, this is made clear by a temporally variable capacity 690 (Cs) connected parallel to the diode 630. Here, according to selection of the diode 630, a physical construction element can be omitted for the temporally variable capacity 690.

A coupling point of the second capacitive element 640 is connected with a coupling point of a resistive element 650, and another coupling point of the second capacitive element 640, which is averted to the coupling point, is connected with another coupling point of the resistive element 650. Here, the resistive element 650 shows a resistivity R₁. A coupling point of the resistive element 650 is contacted via a third capacitive element 660, and another coupling point of the resistive element 650, which is averted to the coupling point, is connected via a fourth capacitive element 670 to a tissue 680 (e.g., muscles or nerves). Here, a tissue impedance amounts to ZG. The third capacitive element 660 shows a capacity C₃, and the fourth capacitive element 670 a capacity C₄.

The parallel resonant circuit 605 gets an energy, e.g., transferred from a resonant circuit of a base station, with a given frequency (HF) and amplitude, which can be generated, for instance, by a resonance amplifier. The energy can be modulated, depending on parameter specifications, for the stimulation, e.g., with the duration of the stimulation. For obtaining the stimulation impulse itself, a demodulation or rectification is carried out by using the diode 630. With an attenuation section formed by the second capacitive element 640 and the resistive element 650, a smoothing occurs, and a potential-free extraction of an induced voltage u(t) occurs in the tissue 680 with the third and fourth capacitive element used in each case as a coupling condenser 660; 670. Here, the time-related induced voltage u(t) causes a time-related flow i(t). According to strength of the induced voltage, the flow varies, and a vibration behaviour of the parallel resonant circuit 605 by the capacity diode 630 depending on the flow, or expressed in other words, the internal frequency of the parallel resonant circuit 605. Hereby, a back-radiated signal can enclose information, which can permit conclusions about, for instance, an internal implant frequency or an impedance of the tissue 680 at a point of the implant 600.

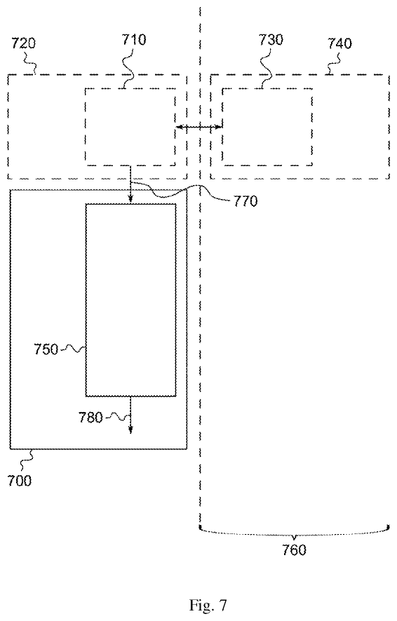

FIG. 7 shows an execution example for an evaluation device 700 for determining the energy transferred from an electric resonant circuit 710 of a base station 720 to a receiver resonant circuit 730 of a medical implant 740. Again, optionally available components are shown with stroked lines and stroked boxes. Here, the receiver resonant circuit 730 can be identical with the parallel resonant circuit 605, and the medical implant 740 with the medical implant 600 (refer to FIG. 6). The evaluation device 700 encloses an analyser 750, which is designed to determine a modulation characteristic of a signal appearing in the electric resonant circuit 710 of the base station 720, and to determine the energy transferred to the receiver resonant circuit 730 based on the modulation characteristic. Here, the medical implant 740 can be inside a human or animal body 760. The resonant circuit 710 and the receiver resonant circuit 730 are coupled with each other wireless, for instance, inductive. Thus an energy can be transferred to the receiver resonant circuit 730 via the resonant circuit 710. Vice versa, a retroactive effect from the implant on the receiver resonant circuit also occurs via the inductive coupling. Further, the resonant circuit 710 is coupled with the analyser 750, so that a transfer 770 of information, for instance, of the signal, can occur from the resonant circuit 710 to the evaluation device 750. The analyser 750 can also be designed to carry out internal or external issue 780 of the ascertained energy with respect to the evaluation device 700. The base station 720 can be coupleable, as shown in FIG. 7, externally with the evaluation device 700, or alternatively enclose the evaluation device 700 and the resonant circuit 710.

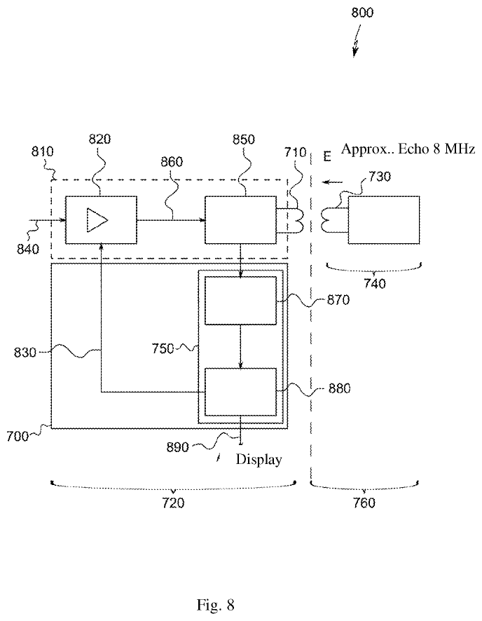

Another block diagram of an evaluation device 700 according to an even more detailed execution example is shown in FIG. 8. Components, which show a correspondence in FIG. 7, are marked with the same reference signs, and are not explained here again. Rather only the differences are mentioned. The base station 720 encloses, in FIG. 8, the evaluation device 700 and a controlled system 810, which is coupled with the resonant circuit 710. The controlled system 810 encloses a resonance amplifier 820, which is designed to receive a control signal 830 from the evaluation device 700, or said more exactly, from the analyser 750, and a reference variable 840, for instance, an input voltage. The resonance amplifier 820 is further designed to change a resonance frequency of the resonant circuit 710 based on the control signal 830. The controlled system further encloses a decoupling section 850, which gets a correcting variable 860, e.g., an excitation voltage or excitation flow of the resonance amplifier 820 and transfer s a decoupled variable to the resonant circuit 710. Besides, about the decoupling section, the signal appearing in the resonant circuit 710 can be transferred to the analyser 750. Furthermore, the resonant circuit 710 can be enclosed by an output stage of the resonance amplifier 820. The signal appearing in the resonant circuit 710 can be caused, for instance, by the receiver resonant circuit 730, and can be an echo signal with a frequency of several Megahertz, e.g., 8 MHz. In some execution examples, the medical implant 740 and the base station 720 can be enclosed by a common system 800.

The analyser 750 further encloses a demodulator 870, which is designed to determine the modulation characteristic of the signal by demodulation. The analyser 750 further encloses a microprocessor 880, which is designed to receive information about the modulation characteristic from the demodulator 870. The microprocessor 880 is further designed to determine the energy transferred to the receiver resonant circuit 730 based on the modulation characteristic. In addition, the microprocessor 880 can be designed to provide a display signal 890 with information about the energy to a display device. The microprocessor 880 or the analyser 750 can also be designed to generate the control signal 830 based on the ascertained energy and to provide to the resonance amplifier 820.

Expressed in other words, the base station 720 can enclose modules for the energy supply and control of the implant 740. Here, the resonance amplifier 820 carries out the energy supply and control of the resonant circuit 710 (or, in other words, a primary coil of the resonant circuit 710). The resonant circuit 710 can be used for the amplifier and also for recording the echo signal. For this, these two functions can be decoupled by means of the decoupling section 850, by which a bidirectional transfer can be enabled. The decoupling section 850 (for instance, made in the form of switch) can also cause a sufficient suppression of a swing-out of the resonant circuit 710 towards the trailing edge of the excitation or stimulation impulse.

The echo signal can be demodulated in an execution example, and the lower-frequency signal resulting from it can be provided to the microprocessor 880. Besides, the microprocessor 880 can carry out an Analogous-Digital conversion. The echo signal can be evaluated, e.g., with a customary display unit or can be used for a regulation. The regulation can be desirable, for instance if a maintaining a defined stimulation under varying local conditions (e.g., variation of the coupling between resonant circuit 710 and receiver resonant circuit 730 as a result of movements or changing tissue impedances) is aimed at. Furthermore, it can be possible to adapt this concept individually in such a way that there are several application possibilities, like for example an easy functional check of the implant 740 or even more complicated measuring functions and monitoring functions.

Alternatively for a use of a demodulator for the demodulation of a frequency-modulated signal, it can also be possible in some execution examples to determine the frequency reflected from the implant directly. For this, for instance, a microprocessor with increased speed can be used, which is designed to work according to a principle of a number frequency meter.

Again explained in other words, a passive implant for the nervous stimulation or muscle stimulation can enclose a receiver resonant circuit and a component for the rectification and smoothing of a voltage to generate a stimulation impulse from an external, high-frequency excitation by the base station. Furthermore, a voltage-limiting construction element, e.g., a Zenerdiode, can be used, by which an over-stimulation can be avoided, and thus an improved protection of a test subject can be possible. Alternatively, rectification and voltage limitation can be done by a capacity diode. The capacity of the diode can change with a voltage adjacent to it. An accessible diode voltage can be given by means of the strength of the control by the base station and by the respective final impedance (tissue impedance) of the passive implant. The developing capacity can thus depend on the stimulation strength and tissue impedance. However, a capacity change in the receiver resonant circuit can also cause a change in the tuning between base station and implant. In case of a wrong tuning by the changing capacity of the receiver resonant circuit, lesser energy can possibly reach to the implant, what can entail a voltage change and thus capacity change in an opposite direction. According to selection of the capacity diode, this interaction can occur between implant and base station in a frequency range, which can differ from the stimulation frequency, for instance, by at least one or also several scales (factor 10 or higher). A modulation of the control can thus occur retrospectively in the resonant circuit of the base station. After the respective demodulation, a modulation characteristic of the echo signal can be made available in the base station with the frequency measured, for instance, by means of a microprocessor. With this, it can be possible to limit the voltage in the implant automatically by using a capacity diode with a certain breakthrough voltage, and thus to allow an improved protection of the test subject.

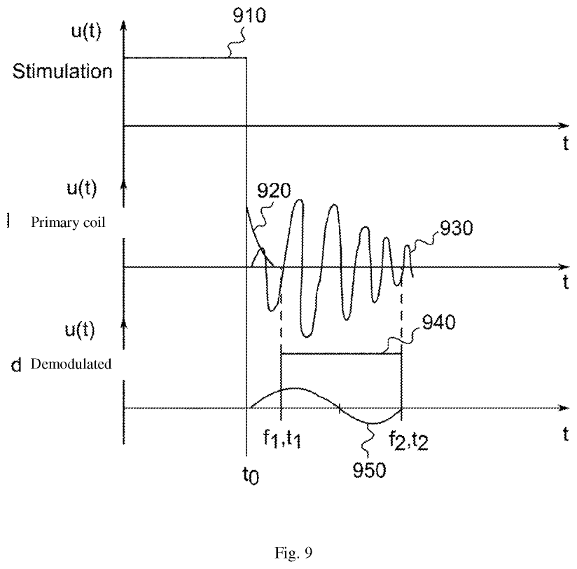

As described above, a remaining energy, which is available immediately after a stimulation impulse or another specific external excitation, can be used in the implant for determining the energy transferred to the medical implant according to the aforementioned first variation. FIG. 9 shows, here, appearing temporal courses of signals, shown here in the form of voltages, in different components of the base station. An external excitation 910 (stimulation) occurs at first in the form of a voltage value, which is constant over a predetermined time (rectangle voltage) and which ends at a time to. In the primary coil of the resonant circuit, a subsiding primary signal 920 as well as the echo signal 930 appears from the time to. In some execution examples, the modulation characteristic of a frequency change corresponds to a frequency modulation of the echo signal 930. For determining the frequency change, the demodulator can be designed to measure a first frequency f1 of the echo signal 930 in a first time t1 and a second frequency f2 in the second time t2. Here, the second time t2 temporally follows the first time t1. The two time points can limit a measuring window 940. Determining the modulation characteristic encloses a determination a difference in the first frequency f1 and the second frequency h. As it becomes evident from FIG. 9, in some execution examples, the evaluation device can be designed to determine the modulation characteristic after transferring the energy (or, in other words, after the time to is exceeded). The measuring window 940 can thus follow temporally or immediately after the time to. The echo signal 930 shows a temporally changing frequency, so that a demodulated signal 950 can be determined from this by the demodulator. The transferred energy depends, in other words, on the magnitude of the difference in the first frequency f1 and the second frequency f2 or a course of the demodulated signal 950.

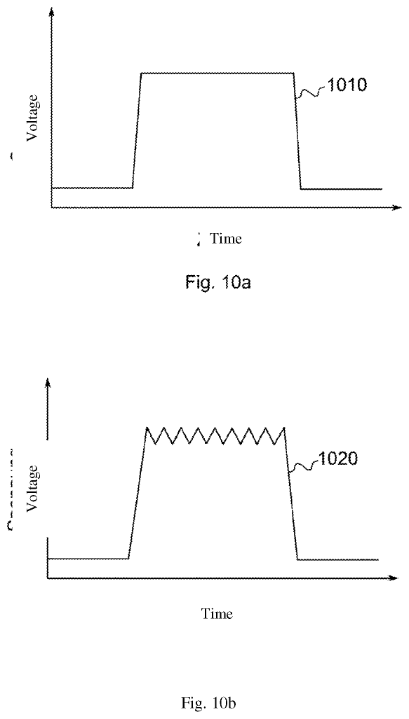

According to the aforementioned second variation, energy can be used, which is supplied to the implant during the stimulation. Here, in other words, the measuring window 940 can coincide temporally with the external excitation 910 at least partially. FIG. 10a shows a temporal course of a customary, approximately rectangular (constant in sections), rectified stimulation impulse 1010. In the comparison with this, FIG. 10b shows a temporal course of a stimulation impulse 1020, which can appear in the application of the second variation in the resonant circuit. In other words, the stimulation impulse 1020 is an amplitude-modulated signal. Again expressed in other words, the stimulation impulse 1020 can show an overlapping of two signals, frequencies of which differ by at least one scale.

In some execution examples, the modulation characteristic of a frequency of an amplitude modulation can correspond to the signal. The demodulator can be further designed to determine an envelope curve of the signal. Here, the modulation characteristic can correspond to a frequency of the envelope curve. The evaluation device can be designed, in some execution examples, to determine the modulation characteristic during the transfer of the energy. By this it can be possible to generate also those frequencies directly, which permit an immediate evaluation with a microprocessor.

By using the second variation, the configured stimulation parameters can be compared with the stimulation parameters appearing in the implant (e.g., stimulation flow) at the same time during a stimulation process and can be readjusted if necessary. Thus a quicker readjustment of the stimulation parameters can be allowed, under circumstances, also during movement of the patient or tilting of the receiver resonant circuit of the implant to the resonant circuit of the base station (or their respective coils to each other). Here, it can be possible to reduce a components required in the implant, since several functions can be taken over by the capacity diode at the same time, which could otherwise require several separate components (e.g., diode for the rectification and an additional Zenerdiode). The capacity diode can be used as a rectifying construction element, and limit, at the same time, the voltage. Here, a higher protection of the patient can be allowed, possibly, by a reduced breakthrough voltage. An evaluation of the signal for the second variation can occur further with the help of a simplified application of electronic components. An amplitude modulation as well as a demodulation occurs in the base station. Here, the excitation frequency can be, for instance, around the 30 times above a frequency of the demodulated signal. Here, the frequency of the demodulated signal can be in the range of some 100 kHz. Measuring this frequency can be already possible with simplified and energy-saving microcontrollers in context of a necessary exactness.

The first and second variation can be applied with the help of the execution example explained in FIG. 8. In other words, this execution example can enclose a control loop. With the control signal 830, the stimulation parameters can be configured, and furthermore, their observance can be monitored with the help of the modulation characteristic. Here, the patient can be protected, since due to the originating frequency of the echo signal in case of too high stimulation energy, an intervention for the reducing the energy can also become possible via the base station 720. By this, the number of necessary, implant-sided components can be possibly reduced.

For the demodulation, a so-called runtime demodulator can be used further, for instance, for a frequency range of 6-10 MHz. This can be formed by digital construction elements, e.g., a NAND gate.

In some execution examples, qualities of the tissue and other surrounding conditions of the implant change only to comparatively bigger time scales. This can entail that with a predetermined cycle, the stimulation parameters can be configured with the help of the anticipated surrounding conditions for the respectively next stimulation with increased exactness. Here, a cycle can enclose, at first, a stimulation (initial excitation), furthermore an echo evaluation and parameter correction, and again a stimulation, echo evaluation and parameter correction, etc. Here, a measurement can show several steps. At first, the implant is excited initially with relatively low energy (echo-receipt mode). With the help of the echo signal, a basic functional check of implant is carried out. Control variables for the energy supply to the implant are configured considering the desired stimulation parameters by means of a microprocessor. A stimulation follows, and, for instance, a renewed receipt of the echo signal at the end of the stimulation impulse. With the help of the echo signal, an examination of or correction in the stimulation parameters can be carried out. Thus the control variables can be configured again.

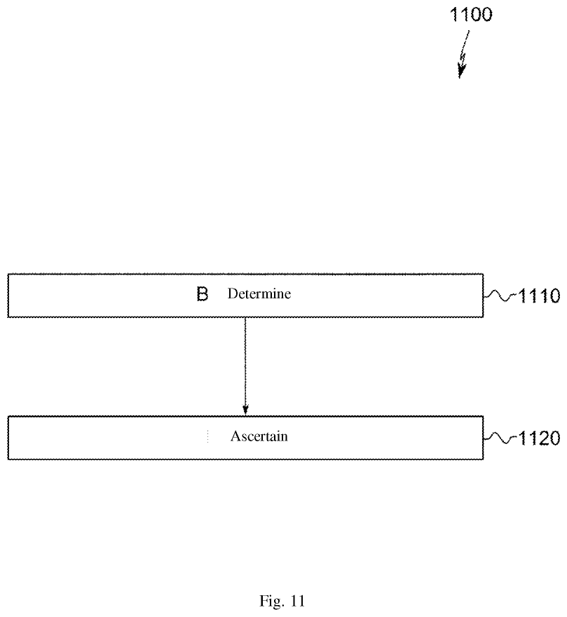

FIG. 11 shows an execution example of a procedure 1100 for determining the energy transferred from an electric resonant circuit of a base station to a receiver resonant circuit of a medical implant. The procedure 1100 encloses determining 1110 of a modulation characteristic of a signal appearing in an electric resonant circuit of the base station. Besides that, the procedure 1100 encloses ascertaining 1120 of an energy transferred to the receiver resonant circuit based on the modulation characteristic. By this it can be enabled to use an echo signal appearing during an energy transfer for obtaining the parameters appearing in the implant. Here, an internal energy supply of the implant can possibly be omitted.

Some execution examples refer further to an active implant, in which an energy memory can be omitted. The implant and the base station can be enclosed by a common system. Here, control loops (Closed-Loop) can be implemented for influencing biological or technical parameters in biological or technical systems. Some execution examples can contribute, possibly, to an increased loading capacity of the system. A continuous or quasi-continuous measurement of parameters and passive reading of the same can occur further. Furthermore, a comparatively high degree of Software can be used for the operation of Closed-Loop systems, what can improve a miniaturization. By an electric measuring process, possibly a measurement in the implant and a more exact configuration possibility of stimulation parameters can be achieved. Here, an additional power requirement for obtaining the echo signal can be reduced or even omitted. Furthermore, a need for electronic construction elements can be possibly reduced. In some execution examples, measuring signals (demodulated signals) can be evaluated by the base station. This can enable an automatic tracking of stimulation parameters in the body of the test subject, simplify an electronic function of the implant, or also improve an intelligence of external electronics. Besides that, an evaluation of the measuring signal could be simplified. Also a functional check of implants could become possible during and after an implantation, whereby an unintentional stimulation can be possibly avoided. Execution examples can be used, for instance, for a functional check of implants, a configuration and control of defined stimulation parameters in the body or determination of a tissue impedance (muscular tissue, fat) in the body.

The features disclosed in the preceding description, the following claims and the enclosed figures can be important individually as well as in any combination for the realization of an execution example in its different forms and they can be implemented.

Although some aspects were described in connection with a device, it is understood that these aspects also show a description of the respective procedure, so that a block or a construction element of a device is also to be understood as an appropriate procedural step or as a feature of a procedural step. Analogous to this, the aspects, which were described in connection with one or as a procedural step, also show a description of a corresponding block or detail or feature of a corresponding device.

According to certain implementation requirements, execution examples of the invention can be implemented in hardware or in software. The implementation can be carried out by using a digital saving medium, for instance, of a floppy disk, a DVD, a Blu-Ray Disc, a CD, a ROM, a PROM, an EPROM, an EEPROM or a FLASH memory, a hard disk or another magnetic or optical memory, in which electronically readable control signals are stored, which can interactor cooperate with a programmable hardware component in such a way that the respective procedure is carried out.

A programmable hardware component can be formed by a processor, a computer processor (CPU=Central Processing Unit), a graphic processor (GPU=Graphics Processing Unit), a computer, a computer system, an integrated switching circuit specific for application (ASIC=Application-Specific Integrated Circuit), an integrated switching circuit (IC=Integrated Circuit), an One-chip system (SOC=system on Chip), a programmable logic element or a field-programmable Gate array with a microprocessor (FPGA=Field Programmable Gate Array).

Hence, the digital saving medium can be read by a machine or computer. Some execution examples enclose a data carrier, which shows electronically readable control signals, which are able to cooperate with a programmable computer system or a programmable hardware component i such a way that one of the procedures described here is carried out. One execution example is thus a data carrier (or a digital saving medium or a computer readable medium), in which the programme for executing one of the procedures described here is recorded.

Generally, execution examples of the present invention can be implemented as a program, firmware, and computer programme or computer programme product with a programme code or as data, whereby the programme code or the data is effective to the extent to carry out one of the procedures, when the programme runs on a processor or a programmable hardware component.

The programme code or the data can be stored, for instance, also on a machine-readable carrier or data carrier. Among the rest, the programme code or the data can be present as a source code, machine code or byte code as well as another intercode.

Another execution example is further a data flow, a signal series or a sequence of signals, which shows or show the programme for carrying out one of the procedures described here. The data flow, the signal series or the sequence of signals can be configured, for instance, to the extent to be transferred via a data communication connection, for instance, via the Internet or another network. Execution examples are thus also data representing signal series, which are suitable for a sending via a network or a data communication connection, whereby the data shows the program.

A programme according to an execution example can implement one of the procedures during its execution, for instance, by the fact that it reads storage locations or writes a date or several dates in this, by which possibly switching procedures or other procedures are activated in transistor structures, in amplifier structures or in other electric, optical, magnetic components or components working according to another functional principle. Accordingly, data, values, sensor values or other information of a programme can be recorded, determined or measured by reading a storage location. Hence, a programme can record, determine or measure variables, values, measurement variables and other information by reading one or several storage locations, as well as cause, arrange or carry out an action by writing in one or several storage locations as well as control other devices, machines and components.

The execution examples described above show only one illustration of the principles of the present invention. It is understood that modifications and variations of the orders and details described here will make sense to other experts. Therefore, it is intended that the invention would be limited only by the protective extent of the following patent claims and not by the specific details, which were presented here with the help of the description and the explanation of the execution examples.

What is claimed is:

1. A closed-loop control circuit for a base station for transmitting energy to a receiver by means of an electrical resonant circuit, comprising:
the electrical resonant circuit;
an evaluation device which is designed to compare energy transmitted to a receiver resonant circuit of the receiver with a target energy value,
wherein the closed-loop control circuit is designed to cause a changed energy input into a receiver resonant circuit of the receiver by changing a resonant frequency of the electrical resonant circuit on the basis of a result of the comparison,
wherein the evaluation device is designed to determine the transmitted energy on the basis of a modulation property of a feedback signal that couples to the electrical resonant circuit,
wherein the closed-loop control circuit is designed to at least partially counteract a change in a coupling of the electrical resonant circuit to the receiver resonant circuit by changing the resonant frequency of the electrical resonant circuit,
wherein the closed-loop control circuit is designed to move the resonant frequency of the electrical resonant circuit away from a natural frequency of the receiver resonant circuit if the transmitted energy is greater than the target energy value,
characterized in that the closed-loop control circuit comprises a resistive element which is coupled to the resonant circuit, has a temporally variable resistivity and comprises an open-loop control connection for receiving an open-loop control signal for changing the resistivity.

2. The closed-loop control circuit according to claim 1, which is designed to bring the resonant frequency of the resonant circuit closer to the natural frequency of the receiver resonant circuit if the transmitted energy is less than the target energy value.

3. The closed-loop control circuit according to claim 1, which is designed to change the resonant frequency by changing an electrical resistance in the resonant circuit.

4. The closed-loop control circuit according to claim 3, which is designed to change the electrical resistance by varying an excitation current, wherein the electrical resistance is an effective resistance of the electrical resonant circuit.

5. The closed-loop control circuit according to claim 4, which is designed to change the electrical resistance within a predetermined interval of time, wherein the interval of time is shorter than an oscillation period of the excitation current.

6. The closed-loop control circuit according to claim 5, which also comprises a series circuit comprising a first electrical component and a second electrical component, wherein the resonant circuit is coupled to an electrically conductive connection between the first electrical component and the second electrical component, with the result that the excitation current is caused by a first input signal from the first electrical component and/or a second input signal from the second electrical component.

7. The closed-loop control circuit according to claim 6, also comprising at least one first voltage source which is designed to generate the first input signal in such a manner that the first input signal alternately has a rising or falling profile in the interval of time and a constant profile in a further interval of time.

8. The closed-loop control circuit according to claim 7, wherein a ratio of the interval of time to the further interval of time is less than 1.

9. The closed-loop control circuit according to claim 7, which is designed to subject a ratio of the interval of time to the further interval of time to closed-loop control.

10. The closed-loop control circuit according to claim 9, which is designed to reduce the resonant frequency by increasing the ratio of the interval of time to the further interval of time or to increase the resonant frequency by reducing the ratio of the interval of time to the further interval of time.

11. The closed-loop control circuit according to claim 7, also comprising at least one second voltage source which is designed to generate the second input signal in such a manner that the second input signal alternately has a rising or falling profile in the interval of time and a constant profile in the further interval of time, wherein the rising profile of the first input signal coincides with the falling profile of the second input signal or the rising profile of the second input signal coincides with the falling profile of the first input signal.

12. The closed-loop control circuit according to claim 6, wherein the first electrical component is a first amplifier, the second electrical component is a second amplifier, the first input signal is a first input voltage and the second input signal is a second input voltage.

13. The closed-loop control circuit according to claim 6, wherein the first electrical component is a first transistor, the second electrical component is a second transistor, the first input signal is a first open-loop control voltage and the second input signal is a second open-loop control voltage.

14. The closed-loop control circuit according to claim 6, which also comprises a series circuit comprising a third electrical component and a fourth electrical component, wherein the resonant circuit couples to a bridge arm between an electrically conductive connection between the first electrical component and the second electrical component and an electrically conductive connection between the third electrical component and the fourth electrical component, with the result that the excitation current is caused by the first input signal, the second input signal, a third input signal from the third electrical component and/or a fourth input signal from the fourth electrical component.

15. The base station having a closed-loop control circuit according to claim 1, wherein the electrical resonant circuit is designed to receive a feedback signal with a modulation property comprising information about the energy transmitted to the receiver resonant circuit of the receiver.

16. A system comprising the receiver and the base station according to claim 15, wherein the receiver is designed to receive an energy signal and to transmit the feedback signal.
